(19) United States
(12) Patent Application Publication
(10) Pub. No.: US 2025/0303090 A1
(43) Pub. Date: Oct. 2, 2025

(54) TRACHEAL COLLAR WITH INTEGRATED SENSORS

(71) Applicant: BioVentures, LLC, Little Rock, AR (US)

(72) Inventors: Thomas Nienaber, Little Rock, AR (US); Adria Abella Villafranca, Little Rock, AR (US); Kevin Sexton, Little Rock, AR (US); Gresham Richter, Little Rock, AR (US); Joseph Sanford, Little Rock, AR (US)

(21) Appl. No.: 18/864,370

(22) PCT Filed: May 10, 2023
(86) PCT No.: PCT/US2023/021727

§ 371 Date: Nov. 8, 2024

Related U.S. Application Data
(60) Provisional application No. 63/340,688, filed on May 11, 2022.

(51) Int. Cl. A61M 16/04; A61B 5/0205

(52) U.S. Cl. CPC A61M 16/0497; A61B 5/02055; A61M 16/0402; A61M 2205/18; A61M 2205/3584; A61M 2205/8212; A61M 2230/06; A61M 2230/20; A61M 2230/30; A61M 2230/42; A61M 2230/50

(57) ABSTRACT

Disclosed herein is a tracheal collar having a device receptacle. The tracheal collar is operable to stabilize the tracheostomy tube of the patient when the collar is secured around the neck of the patient. A monitoring device is operable to be inserted into the device receptacle. The monitoring device includes a microcontroller and one or more sensors operable to monitor one or more physiological parameters of the patient when the monitoring device is inserted into the device receptacle of the tracheal collar.

### FIELD

The present disclosure relates to a tracheal collar with integrated sensors for monitoring physiological parameters of a patient and methods of use thereof.

### BACKGROUND

Historically, securement devices such as collars have been used to stabilize the tracheostomy (trach) tube of patients who require a surgical tracheostomy. Traditionally, these securement devices have consisted of disposable cloth and Velcro. In addition to the tracheostomy tube and the related securement device, supplementary equipment is required to monitor various physiological parameters of the patient. For example, clinicians monitor respiratory rate and blood oxygen saturation through separate systems to guide mechanical ventilator settings. Additionally, clinicians monitor the heart rate and temperature of patients that require mechanical ventilation. The number of devices needed to monitor these physiological conditions is cumbersome.

Therefore, there is a need for a tracheal collar with integrated sensors to monitor physiological parameters of the patient while reducing the number of disparate sensors, leads, and devices currently used by patients with surgical tracheostomies.

### SUMMARY

This disclosure provides a tracheal collar with integrated sensors and methods of use thereof. One aspect of the present disclosure encompasses a system for monitoring a patient with a tracheostomy tube that includes a collar having a device receptacle. The collar is operable to stabilize the tracheostomy tube of the patient when the collar is secured around a neck of the patient. The system also includes a monitoring device, which includes a microcontroller and one or more sensors, operable to be inserted into the device receptacle of the collar. The one or more sensors are operable to monitor one or more physiological parameters of the patient when the monitoring device is inserted into the device receptacle of the collar.

The collar may be adjustable to secure around various neck sizes and orient the device receptacle to position the one or more sensors near a carotid artery of the patient when the collar is secured around the neck of the patient. The one or more physiological parameters of the patient may be at least one of temperature, heart rate, respiratory rate, blood pressure, and blood oxygen saturation. The one or more sensors may be at least one of a piezoelectric sensor, a photoplethysmographic sensor, an infrared sensor, and a temperature sensor. The microcontroller may be operable to communicate the one or more physiological parameters to a secondary device. The microcontroller may include a wireless transceiver operable for wireless communication including at least one of radio frequency (RF), Wi-Fi, or Bluetooth. The monitoring device may include a rechargeable battery. The collar may be made of an elastomer, such as silicone.

Another aspect of the present disclosure encompasses a system for monitoring a patient with a tracheostomy tube that includes a neck flange having a device receptacle. The neck flange is operable to contact the neck of a patient and to allow a proximal end of the tracheostomy tube of the patient to pass through the void in the neck flange. A tracheostomy collar connects to each end of the neck flange and secures around the neck of the patient so that the tracheostomy collar maintains the position of the neck flange and the neck flange stabilizes the tracheostomy tube of the patient. The system also includes a monitoring device, including a microcontroller and one or more sensors, operable to be inserted into the device receptacle of the neck flange. The one or more sensors are operable to monitor one or more physiological parameters of the patient when the device is inserted into the device receptacle of the neck flange.

Another aspect of the present disclosure encompasses a tracheostomy tube that includes a tube body, a flange extending radially from the tube body, and one or more sensors integrated into the flange of the tracheostomy tube. The flange is operable to contact the neck of a patient and the one or more sensors are operable to monitor one or more physiological parameters of the patient.

Another aspect of the present disclosure encompasses a system for monitoring a patient with a tracheostomy tube that includes a securement apparatus having a device receptacle. The securement apparatus is operable to stabilize the tracheostomy tube of the patient when the securement apparatus is secured around a neck of the patient. The system also includes a monitoring device, which includes a microcontroller and one or more sensors, operable to be inserted into the device receptacle of the collar. The one or more sensors are operable to monitor one or more physiological parameters of the patient when the monitoring device is inserted into the device receptacle of the securement apparatus.

The securement apparatus may be adjustable to secure around various neck sizes and orient the device receptacle to position the one or more sensors near a carotid artery of the patient when the securement apparatus is secured around the neck of the patient. The securement apparatus may be a collar. The collar may include a first section having at least one aperture, a second section having a boss and the device receptacle located adjacent to the boss of the second section, and a third section having at least one aperture and at least one boss. The at least one aperture of the first section may be configured to removably couple to the boss of the third section. The at least one aperture of the third section may be configured to removably couple to the boss of the second section.

The securement apparatus may be a neck flange. The neck flange may include two apertures on opposite sides of the device receptacle, a void operable to allow a proximal end of the tracheostomy tube of the patient to pass through the void in the neck flange. The two apertures may each be operable to receive a collar configured to secure the neck flange around the neck of the patient.

The securement apparatus may be a flange extending radially from a tube body of the tracheostomy tube. The flange may be operable to contact the neck of the patient.

The one or more physiological parameters of the patient may be at least one of temperature, heart rate, respiratory rate, blood pressure, and blood oxygen saturation. The one or more sensors may be at least one of a piezoelectric sensor, an optoelectronic sensor, a photoplethysmographic sensor, an infrared sensor, a pulse oximeter, and a temperature sensor. The microcontroller may be operable to communicate the one or more physiological parameters to a secondary device. The microcontroller may include a wireless transceiver for wireless communication including at least one of radio frequency (RF), Wi-Fi, or Bluetooth. The monitoring device may include a rechargeable battery. The securement apparatus may be made of an elastomer such as silicone.

Also disclosed herein is a method for monitoring a patient with a tracheostomy tube. The method may include inserting a monitoring device into a device receptacle of a tracheostomy collar, the monitoring device comprising a microcontroller and one or more sensors; securing the tracheostomy collar around a neck of the patient to stabilize the tracheostomy tube of the patient; monitoring, via the one or more sensors, one or more physiological parameters of the patient; and communicating, via the microcontroller, the one or more physiological parameters to a secondary device.

The one or more physiological parameters may be respiratory rate, blood oxygen saturation, heart rate, blood pressure, and temperature. The method may further include providing an alarm when one or more physiological parameters exceed a threshold. The method may further include analyzing historic data from the one or more physiological parameters to determine further treatment options. The microcontroller may communicate the one or more physiological parameters via a wireless transceiver operable for wireless communication including at least one of radio frequency (RF), Wi-Fi, or Bluetooth. The one or more sensors may be at least one of a piezoelectric sensor, an optoelectronic sensor, a photoplethysmographic sensor, an infrared sensor, a pulse oximeter, and a temperature sensor.

Another aspect of the present disclosure encompasses a tracheostomy tube. The tracheostomy tube may include a tube body, a flange extending radially from the tube body having a device receptacle, and a monitoring device. The flange may be operable to contact a neck of the patient. The monitoring device may be operable to be inserted into the device receptacle. The monitoring device may have a microcontroller and one or more sensors. The one or more sensors may be operable to monitor one or more physiological parameters of the patient.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1A is a perspective view of an adjustable tracheostomy collar with a device receptacle to receive a monitoring device.

FIG. 1B is a top view of the adjustable tracheostomy collar with a device receptacle to receive a monitoring device.

FIG. 1C is a front view of the adjustable tracheostomy collar.

FIG. 1D is a bottom view of the adjustable tracheostomy collar.

FIG. 1E is a perspective view of the device receptacle to receive a monitoring device.

FIG. 1F is a top view of the device receptacle to receive a monitoring device.

FIG. 2A is a perspective view of a neck flange, containing a flange base with a device receptacle to receive a monitoring device and a flange cover, configured to be connected to a tracheostomy collar.

FIG. 2B is a top view of the flange base of the neck flange configured to be connected to a tracheostomy collar.

FIG. 2C is a bottom view of the flange base configured to be connected to a tracheostomy collar.

FIG. 2D is a perspective view of the device receptacle to receive a monitoring device within the flange base.

FIG. 2E is a top view of the device receptacle to receive a monitoring device within the flange base.

FIG. 2F is a perspective view of the flange cover of the neck flange configured to be connected to a tracheostomy collar.

FIG. 2G is a bottom view of the flange cover configured to be connected to a tracheostomy collar.

FIG. 3 is a schematic of a wiring diagram for one embodiment of a monitoring device to monitor physiological parameters of a patient.

FIG. 4 is a photograph of a tracheostomy collar with an infrared sensor (IR) connected to a microcontroller.

FIG. 5A is sample pulse oximetry data from a collar with a sensor placed on skin.

FIG. 5B is sample data, including heart rate and pulse oximetry, from an infrared (IR) sensor on a collar.

FIG. 5C is waveform data from a sensor on tracheostomy collar, showing heart rate with a similar appearance to an electrocardiogram.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

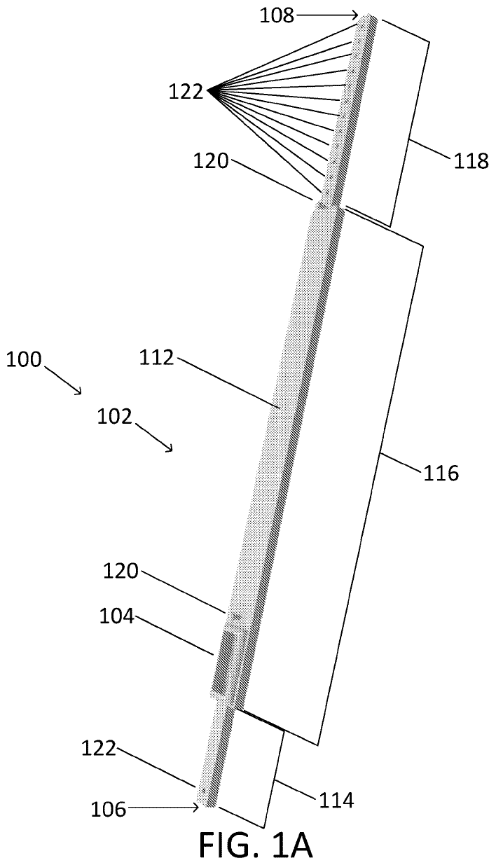

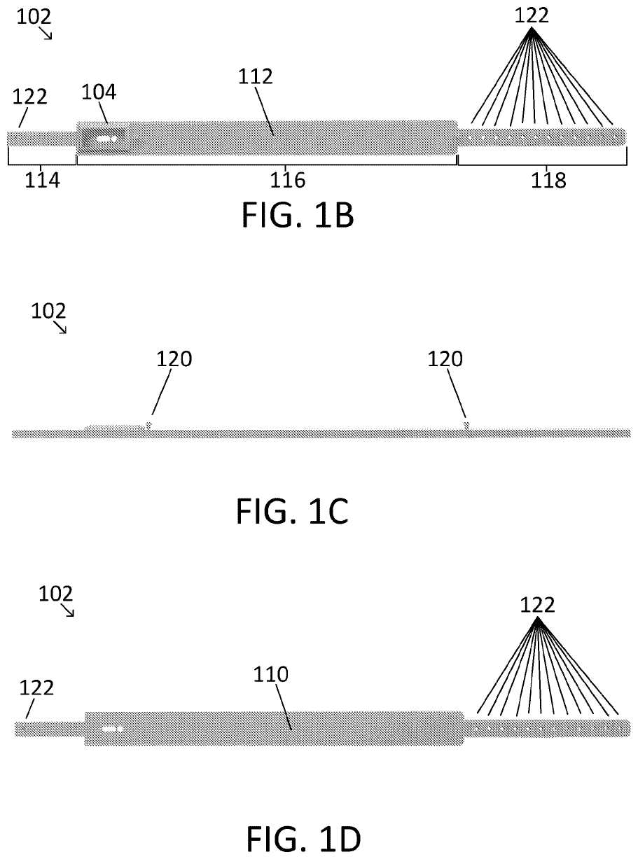

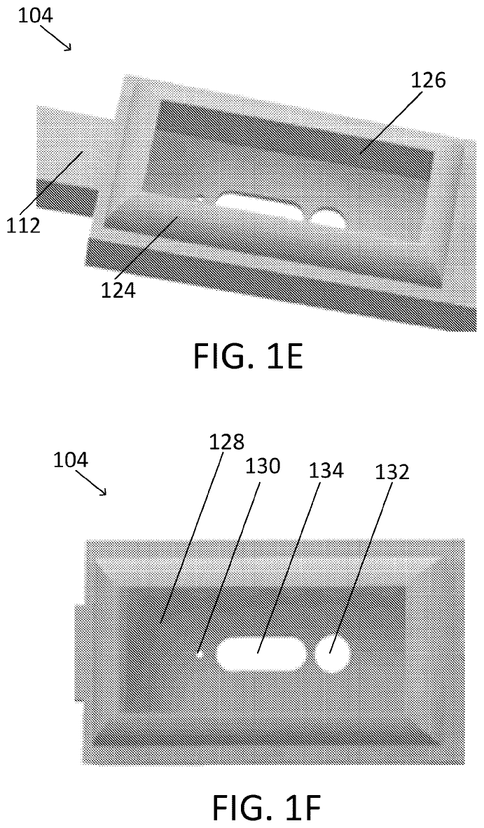

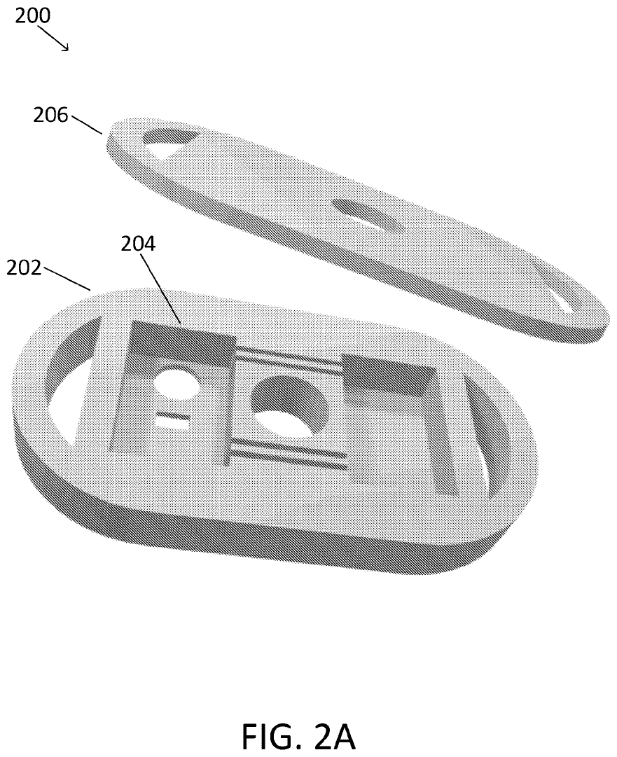

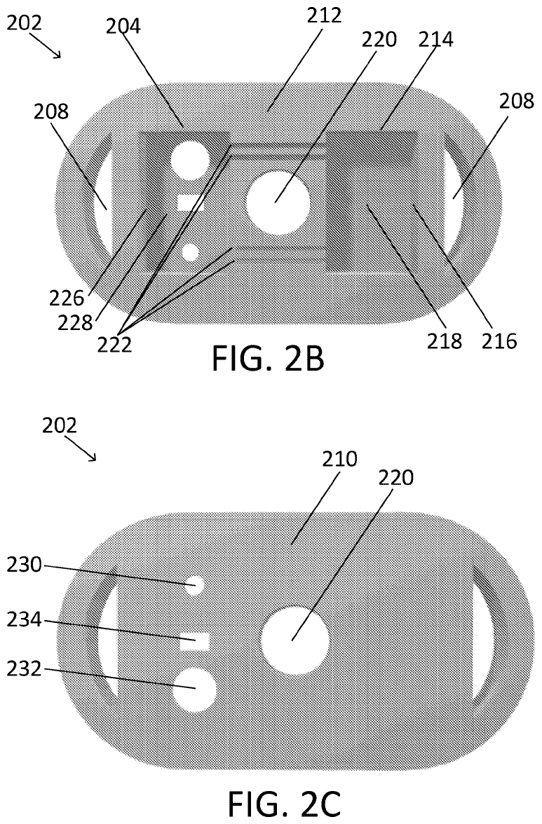

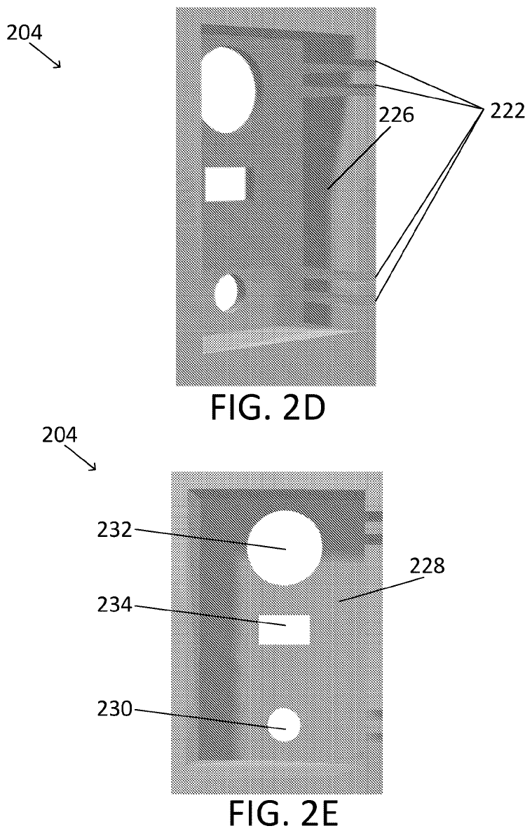

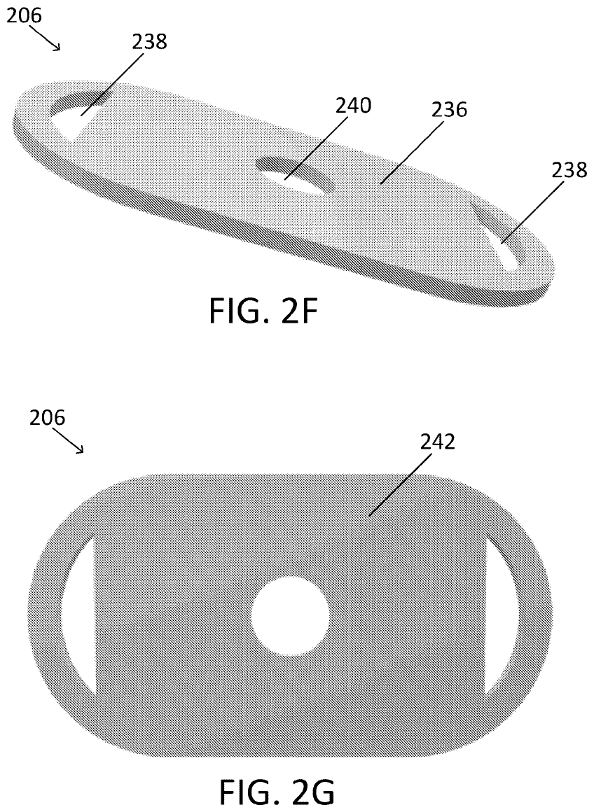

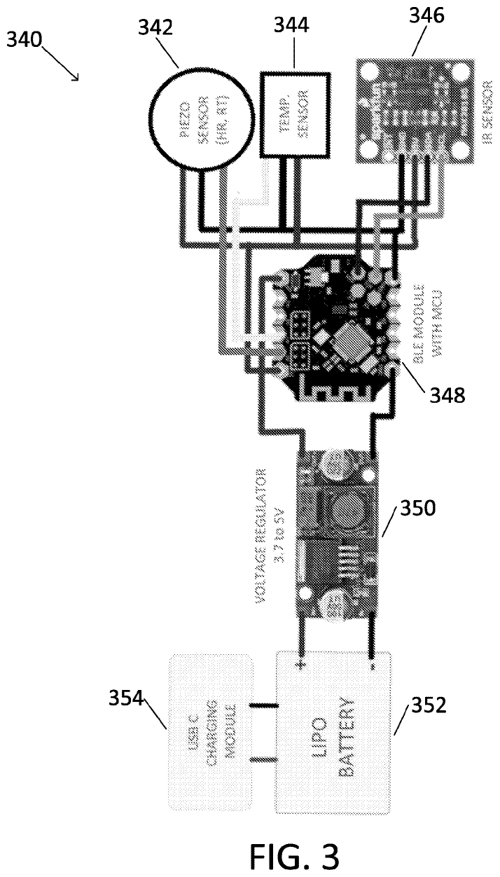

Provided herein is a securement apparatus with integrated sensors and methods of use thereof to improve the monitoring a patient with a tracheostomy tube. As illustrated in FIGS. 1A-1D, the securement apparatus may be a tracheal collar comprising a collar body that includes a device receptacle to receive a monitoring device. In other embodiments, as seen in FIGS. 2A-2E, the securement apparatus may be a neck flange comprising a flange base and a flange cover that is configured to attach to a traditional securement device. In other embodiments, the securement apparatus may be a flange integrated with the tracheostomy tube. For example, the monitoring device may be integrated into the flange extending radially from the tracheostomy tube body. In one embodiment, the monitoring device may include components that are electrically connected as shown in FIG. 3. The monitoring device may be configured to monitor the physiological parameters or biometrics of a patient.

The tracheal collar with integrated sensors may have significant advantages over traditional securement devices that are currently used to secure tracheostomy tubes to patients. Patients, their caregivers, and clinicians desire better tools at home and in the hospital to monitor physiologic changes in the patient. Patients want ease of use, decreased number of devices, portability, and good support from the community. Similarly, clinicians want cost-effectiveness, ease of use, compatibility, and remote communication. The tracheal collar with integrated sensors may achieve these objectives.

The tracheal collar with integrated sensors may be a reusable collar made of an elastic and comfortable material that can be washed, easily disinfected, and may minimize pressure injuries. Conversely, traditional tracheostomy securement devices are disposable and consist of cloth collars with cushion material that are secured around the patient's neck and attached to the tracheostomy tube via Velcro, fabric twill, or a clip.

The tracheal collar with integrated sensors may provide safety by monitoring the physiological parameters of patients with tracheostomy tubes. Tracheostomy tubes are necessary in adults and children who require prolonged ventilation or protection from severe upper airway obstruction. In the pediatric population, once a tracheostomy is placed, the time to decannulation is often measured in years. Unlike adults, most children are less able to report respiratory difficulty, accidental decannulation, or changing health status due either to age or to developmental delays. Thus, these patients require a host of equipment to assist in physiological monitoring apart from the actual tracheostomy itself.

The tracheal collar with integrated sensors may decrease the number of disparate sensors, leads, and devices currently used to monitor patients with tracheostomy tubes. Mechanical ventilation requires the use of a tracheal tube. To guide ventilator settings, respiratory parameters such as respiratory rate and pulse oximetry are currently monitored through separate and attached systems. The pulse oximeter, for example, is secured around a finger, toe, or hand of the patient. Additionally, heart rate and temperature assist clinicians in caring for patients requiring mechanical ventilation. Thus, the number of devices currently needed to perform this monitoring at home is cumbersome and difficult.

The tracheal collar with integrated sensors may achieve wireless physiological monitoring of patients with tracheostomy tubes while providing enhanced mobility for the patients. As noted above, a pulse oximeter probe is most often attached to a finger or toe. This probe is often poorly secured or poorly tolerated by pediatric patients. Moreover, these probes limit mobility of the extremity of which it is attached. Separately, a thermometer is required to check temperature. These devices are all separate, have their own alarms, do not communicate with each other, are not recorded, are inaccessible to remote physicians and are variably able to be used by clinicians.

The tracheal collar with integrated sensors is able to measure physiological parameters (temperature, heart rate, respiratory rate, blood pressure, and blood oxygen saturation) in patients with or without mechanical ventilation. Therefore, the system decreases the number of required devices and provides remote monitoring of chronic and acute events in trach-dependent patients, especially at home. The system provides a patient-centered, minimalist, easy to use, robust, portable monitoring device for patients requiring mechanical ventilation. The tracheal collar with integrated sensors may be used for in-home or in-hospital patients. Using wireless technology, such as Bluetooth Low Energy, all of these parameters can be recorded, displayed, and easily viewed by any remote caregiver using a separate electronic device with a screen. Additionally, the system can be configured to provide alerts of acute and life-threatening events.

The monitoring device may be less obtrusive and cumbersome for patients and their caregivers while providing remote alerts and capturing of events. The tracheal collar rests on the skin and approximates the major sources of physiologic activity (i.e. blood vessels such as the carotid artery and jugular vein). Thus, sensors can be incorporated into the tracheal collar to monitor physiological parameters while limiting the footprint of the device. The monitoring device may include piezoelectric, photoplethysmographic, and temperature sensors, and the device may be inserted into a device receptacle of either a tracheal collar or of a neck flange. Piezoelectric sensors are capable of transcutaneously detecting small changes in pressure such as heart rate. Photoplethysmography can monitor oxygen saturations and validate heart rate readings. Thus, the monitoring device decreases the number of disparate sensors, leads, and devices currently used to monitor the physiological parameters of patients with tracheostomy tubes.

FIGS. 1A-1D illustrate the tracheal collar assembly 100. The tracheal collar assembly 100 comprises a collar body 102 that is an elongated strap defining a first end 106, a second end 108, an inner surface 110, an outer surface 112, and a longitudinal axis. The collar body 102 is flexible. The longitudinal axis is substantially straight as illustrated in the figures; however, when the collar body 102 is curved to secure around the neck of a patient, the longitudinal axis will similarly vary. The collar body 102 may contain a generally rectangular or generally square cross section. When the tracheal collar assembly 100 is secured around the neck of a patient, the inner surface 110 of the collar body 102 is in contact with the neck of the patient and the outer surface 112 of the collar body 102 faces outward, away from the neck of the patient.

The collar body 102 includes a device receptacle 104. The device receptacle 104 is configured to receive or accept a monitoring device (not shown in FIGS. 1A-1F). In other words, the collar body 102 is secured around the neck of a patient and a monitoring device is inserted into the device receptacle 104 to monitor various physiological parameters of the patient. The device receptacle 104 is positioned on the collar body 102 so that the device receptacle 104 is located near the carotid artery of a patient when the tracheal collar assembly 100 is secured around the neck of a patient. In other words, when the tracheal collar assembly 100 is secured around the neck of a patient, the device receptacle 104 and the associated monitoring device (not shown in FIGS. 1A-1F) will be positioned at or near a carotid artery of a patient. In one embodiment, the device receptacle 104 is located within a second section 116 of the collar body 102, adjacent to the first section 114 of the collar body 102.

The collar body 102 may contain a first section 114, a second section 116, and a third section 118. In some embodiments, the first section 114, second section 116, and third section 118 are manufactured as one piece. The distance between the inner surface 110 and outer surface 112 defines a thickness of the collar body 102. In one embodiment, the thickness of the first section 114, the second section 116, and the third section 118 of the collar body 102 are the same. In other examples, the thickness of the first section 114, the second section 116, and the third section 118 of the collar body 102 may be different. In one embodiment, the width of the first section 114, the second section 116, and the third section 118 of the collar body 102 are different. For example, the width of the first section 114 and the third section 118 of the collar body 102 may be narrower than the width of the second section 116. In other examples, the width of the first section 114, the second section 116, and the third section 118 of the collar body 102 may be the same.

The collar body 102 may contain one or more apertures 122 that extend through the collar body 102. Each aperture 122 may be generally circular in shape, thereby forming a generally cylindrical void through the thickness of the collar body 102. In one embodiment, a plurality of apertures 122 extends through the third section 118 of the collar body 102 and one aperture 122 extends through the first section 114, near the first end 106 of the collar body 102. The apertures 122 may be configured to accept bosses 120 protruding for the collar body 102 in order to secure the collar body around the neck of a patient (e.g., the apertures 122 may be removably coupled to the bosses 120). The plurality of apertures 122 within the third section 118 may be evenly spaced along the longitudinal axis of the collar body 102, whereby the spacing allows for the collar body 102 to be adjustable in order to secure around various patient neck sizes. The collar body 102 is configured so that the device receptacle 104 will be located near the carotid artery of a patient when the tracheal collar assembly 100 is secured around the neck of a patient, regardless of the adjustments necessary (i.e., the specific aperture 122 into which a boss 120 is inserted) to secure the adjustable collar body 102 around the neck of the patient. In other words, when the tracheal collar assembly 100 is secured around the neck of a patient, the device receptacle 104 and the associated monitoring device (not shown in FIGS. 1A-1F) will be positioned at or near a carotid artery of a patient.

The collar body 102 may contain one or more bosses 120. The bosses 120 protrude from the collar body 102 and, in some embodiments, may be generally cylindrical in shape. In one embodiment, one boss 120 is located adjacent to the device receptacle 104 in the second section 116 of the collar body 102 and one boss 120 is located near the interface of the second section 116 and the third section 118. The shape of the bosses 120 and the shape of the apertures 122 correspond, so that a boss can be inserted through an aperture 122. In other words, the apertures 122 are configured to accept the bosses 120. In some embodiments, each boss 120 may have a slightly larger radius at an upper portion of the boss 120 (i.e., near the top surface of the boss 120) than the radius of a lower portion of the boss 120. This larger radius at the upper portion of the boss 120 may be a slightly smaller, same, or slightly larger diameter than the radius of the apertures 122. Similarly, the height of the lower portion of the bosses 120 may be the same or slightly greater than the thickness of the collar body 102 at the aperture 122. This configuration allows a boss 120 to snap into an aperture 122 and secure the collar body 102 around the neck of the patient.

In one embodiment, the collar body 102 is secured around the neck of the patient and attached to a traditional tracheostomy flange. The inner surface 110 of the collar body 102 is placed against the posterior and sides of the neck of the patient. The first end 106 of the collar body 102 is inserted through the back side of a connection aperture at one end of a traditional tracheostomy flange. The first section 114 of the collar body 102 is advanced through the connection aperture and then the first end 106 is folded back onto the collar body 102 so that the outer surface 112 of the first section 114 approaches the outer surface 112 of the second section 116. The aperture 122 located near the first end 106 of the collar body 102 is then snapped into place by receiving the boss 120 located adjacent to the device receptacle 104 in the second section 116 of the collar body 102. The length of the first section 114, the location of the aperture 122 near the first end 106, and the location of the boss 120 adjacent to the device receptacle 104 in the second section 116 are all configured so that the device receptacle 104 and the associated monitoring device will be positioned at or near a carotid artery of the patient.

In the same embodiment, the second end 108 of the collar body 102 is then inserted through the back side of the connection aperture at the other end of the traditional tracheostomy flange. The third section 118 of the collar body 102 is advanced through the connection aperture and then the second end 108 is folded back onto the collar body 102 so that the outer surface 112 of the third section 118 approaches the outer surface 112 of the second section 116. One of the plurality of apertures 122 located within the third section 118 of the collar body 102 is then snapped into place by receiving the boss 120 located near the interface of the second section 116 and the third section 118. The one aperture 122 is selected, from among the plurality of apertures 122 in the third section 118, to provide a proper fit for the respective neck size of the patient. The length of the third section 118, the location of the plurality of apertures 122 located within the third section 118, and the location of the boss 120 near the interface of the second section 116 and the third section 118 are all configured so that the collar body 102 is adjustable to properly secure the tracheal collar assembly 100 to any patient neck size.

As illustrated in FIGS. 1E-1F, the collar body 102 may contain a device receptacle 104. In one embodiment, the device receptacle 104 is generally rectangular in shape. In other embodiments, the device receptacle may be generally circular, ovular, triangular, or square. The device receptacle 104 is defined by sidewalls 126 and a bottom surface 128. The device receptacle 104 may contain one or more apertures 130, 132, 134 through the bottom surface 128 of the device receptacle 104. The one or more apertures 130, 132, 134 may be configured to allow the monitoring device (not shown in FIGS. 1E-1F) to communicate with the neck of the patient. In other words, the one or more apertures 130, 132, 134 may allow one or more sensors of the monitoring device to measure one or more physiological parameters of the patient. In one embodiment, a smaller circular aperture 130, a larger circular aperture 132, and a slotted aperture 134 create openings through the bottom surface 128 of the device receptacle 104. In other examples, the one or more apertures may be a generally ovular shape, triangular shape, square shape, or rectangular shape.

In one embodiment, the device receptacle 104 may contain a raised edge 124 around its perimeter on the collar body 102. The raised edge 124 of the device receptacle 104 defines an inner surface that is the sidewalls 126 for the device receptacle 104. In some embodiments, the raised edge 124 may contain a non-flat profile at the outer perimeter of the raised edge 124. For example, the raised edge may contain a rounded profile, a beveled profile, or a combination thereof. In some embodiments, the raised edge may contain an eased edge profile, a ¼ bevel profile, a ¼ round profile, a ½ bevel profile, a ½ bullnose profile, a mitred profile, a double beveled profile, or a triple beveled profile. In other embodiments, the raised edge may contain a flat or straight edge profile. The height of the raised edge 124 may be configured to achieve the desired thickness of the device receptacle 104.

In one embodiment, the device receptacle 104 is recessed into the collar body 102 in addition to the raised edge 124 around the perimeter of the device receptacle 104. In other words, the collar body 102 may have less thickness at the device receptacle 104 (i.e., the thickness of the collar body at the device receptacle 104 as measured from the bottom surface 128 of the device receptacle 104 to the inner surface 110 of the collar body 102) than the thickness of the collar body 102 apart from the device receptacle 104. The sidewalls 126 of the recessed portion of the device receptacle 104 may coincide with the sidewalls 126 created by the raised edge 124 of the device receptacle 104 in order to form continuous sidewalls 126 around the perimeter of the device receptacle 104. In other embodiments, the device receptacle may be formed by recess in the collar body without a raised edge. In still other embodiments, the device receptacle may be formed by a raised edge without a recess in the collar body. In other words, the collar body may have the same thickness at the device receptacle (i.e., the thickness of the collar body at the device receptacle as measured from the bottom surface of the device receptacle to the inner surface of the collar body) as the thickness of the collar body apart from the device receptacle.

The collar body 102 may be made of a comfortable material, such as an elastic material. The material of the collar body 102 may be reusable, washable to be easily disinfected, and configured to minimize pressure injuries. In some embodiments, the tracheal collar may be made of an elastomer. For example, the tracheal collar may be made of silicone. The collar body 102 may be manufactured by injection molding.

FIG. 2A illustrates a neck flange assembly 200. The neck flange assembly 200 comprises a flange base 202 containing a device receptacle 204. The neck flange assembly 200 further comprises a flange cover 206, which can be joined with the flange base 202 to enclose the monitoring device (not shown in FIGS. 2A-2G) within the device receptacle 204.

As illustrated in FIGS. 2B-C, the flange base 202 defines an inner surface 210, an outer surface 212, and a longitudinal axis. When the neck flange assembly 200 is secured around the neck of a patient, the inner surface 210 of the flange base 202 is in contact with the neck of the patient and the outer surface 212 of the flange base 202 faces outward, away from the neck of the patient. In some embodiments, the inner surface 210 of the flange base 202 is flat and defines a planar surface. In other examples, the inner surface may be curved or contoured in accordance with the shape of the anterior aspect of the neck of a patient. The flange base 202 contains a central opening 220 configured to accommodate a tracheostomy tube of the patient. In other words, the proximal end of the tracheostomy tube of the patient passes through the central opening 220, thereby stabilizing the tracheostomy tube.

The flange base 202 includes a device receptacle 204. The device receptacle 204 is configured to receive or accept a monitoring device (not shown in FIGS. 2A-2G). In other words, a monitoring device is inserted into the device receptacle 204 and the neck flange assembly 200 is secured around the neck of a patient to monitor various physiological parameters of the patient. The device receptacle 204 is positioned along the flange base 202 so that the device receptacle 204 is located near the carotid artery of a patient when the neck flange assembly 200 is secured around the neck of a patient. In other words, when the neck flange assembly 200 is secured around the neck of a patient, the device receptacle 204 and the associated monitoring device (not shown in FIGS. 2A-2G) will be positioned at or near a carotid artery of the patient.

The flange base 202 may contain connection apertures 208, extending through the body of the flange base 202 and located near each end of the flange base 202. The connection apertures 208 are configured to allow a traditional securement device to be attached to the neck flange assembly 200. The traditional securement device may be secured around the posterior and sides of the neck of a patient and the ends of the traditional securement device may be connected to the connection apertures 208 of the flange base 202, which is positioned at the tracheostomy tube at the anterior of the neck of the patient. In some embodiments, the connection apertures 208 may be generally semi-circular in shape. In other examples, the connection apertures may be generally circular, ovular, rectangular, or square. The connection apertures 208 may be on opposite sides of the device receptacle 204. In some embodiments, the traditional securement device may be cloth that is secured around the patient's neck and attached to the flange base 202 at the connection apertures 208 via Velcro, fabric twill, or a clip.

The flange base 202 may contain a second receptacle 214. In one embodiment, the second receptacle is generally rectangular in shape. In other embodiments, the device receptacle may be generally circular, ovular, triangular, or square. The second receptacle 214 is defined by sidewalls 216 and a bottom surface 218 of the second receptacle 214. The second receptacle may contain one or more apertures through the bottom surface of the second receptacle. For example, the one or more apertures may be a generally circular shape, ovular shape, triangular shape, square shape, rectangular shape, or slotted shape to create one or more openings through the bottom surface of the second receptacle.

In some embodiments, one or more communication channels 222 may connect the device receptacle 204 with the second receptacle 214 within the flange base 202. In other words, the one or more communication channels 222 establish connection and communication between the device receptacle 204 and the second receptacle 214. For example, the communication channels 222 may allow electrical wiring to pass between the device receptacle 204 with the second receptacle 214. Each bottom of the one or more communication channels 222 may be coplanar with the bottom surface 228 of the device receptacle 204 and coplanar with the bottom surface 218 of the second receptacle 214.

As illustrated in FIGS. 2D-E, the flange base 202 may contain a device receptacle 204. In one embodiment, the device receptacle 204 is generally rectangular in shape. In other embodiments, the device receptacle may be generally circular, ovular, triangular, or square. The device receptacle 204 is defined by sidewalls 226 and a bottom surface 228 of the device receptacle 204. The device receptacle 204 may contain one or more apertures 230, 232, 234 through the bottom surface 228 of the device receptacle 204. The one or more apertures 230, 232, 234 may be configured to allow the monitoring device (not shown in FIGS. 2A-2G) to communicate with the neck of the patient. In other words, the one or more apertures 230, 232, 234 may allow one or more sensors of the monitoring device to measure one or more physiological parameters of the patient. In some embodiments, a smaller circular aperture 230, a larger circular aperture 232, and a rectangular aperture 234 create openings through the bottom surface 228 of the device receptacle 204. In other examples, the one or more apertures may be a generally ovular shape, triangular shape, square shape, or slotted shape.

As illustrated in FIGS. 2F-G, the flange cover 206 defines an inner surface 242, an outer surface 236, and a longitudinal axis. When the neck flange assembly 200 is assembled, the inner surface 242 of the flange cover 206 is in contact with the outer surface 212 of the flange base 202. Thus, when the neck flange assembly 200 is secured around the neck of a patient, the outer surface 236 of the flange cover 206 faces outward, away from the neck of the patient. The flange cover 206 contains a central opening 240 configured to accommodate a tracheostomy tube of the patient. In other words, the proximal end of the tracheostomy tube of the patient passes through the central opening 240, thereby stabilizing the tracheostomy tube. When the neck flange assembly 200 is assembled, the central opening 240 of the flange cover 206 is in alignment with the central opening 220 of the flange base 202. The proximal end of the tracheostomy tube of the patient can pass through both the central opening 220 of the flange base 202 and the central opening 240 of the flange cover 206.

The flange cover 206 may contain connection apertures 238, extending through the body of the flange cover 206 and located near each end of the flange cover 206. When the neck flange assembly 200 is assembled, the connection apertures 238 of the flange cover 206 are in alignment with the connection apertures 208 of the flange base 202. The connection apertures 208 of the flange base 202 and the connection apertures 238 of the flange cover 206 are configured to allow a traditional securement device to be attached to the neck flange assembly 200. The traditional securement device may be secured around the posterior and sides of the neck of a patient and the ends of the traditional securement device may be connected to the connection apertures 208 of the flange base 202 and the connection apertures 238 of the flange cover 206, which is positioned at the tracheostomy tube at the anterior of the neck of the patient. In some embodiments, the connection apertures 238 of the flange cover 206 may be generally semi-circular in shape. In other examples, the connection apertures may be generally circular, ovular, rectangular, or square. In some embodiments, the traditional securement device may be cloth that is secured around the patient's neck and attached to the neck flange assembly 200 at the connection apertures 208, 238 via Velcro, fabric twill, or a clip. The traditional securement device may be modified as necessary to attach to the neck flange assembly 200.

The flange base 202 and flange cover 206 may be made of a comfortable material, such as an elastic material. The material of the flange base 202 and flange cover 206 may be reusable, washable to be easily disinfected, and configured to minimize pressure injuries. In some embodiments, the flange base and flange cover may be made of an elastomer. The flange base 202 and flange cover 206 may be manufactured by injection molding.

In other embodiments, monitoring device may be integrated into the tracheostomy tube body. For example, the sensors may be integrated into the flanges of the tracheostomy tube body. In another example, the tracheostomy tube body may have a device receptacle in the flange of the tracheostomy tube body. The device receptacle may be operable to receive the monitoring device. The flange may be operable to position the monitoring device near a carotid artery of the patient. The battery may be housed in the collar, with conductive connectors.

As illustrated in FIG. 3, the monitoring device 340 is configured to monitor one or more physiological parameters of the patient. The monitoring device is operable to be inserted into the device receptacle 104 of the tracheal collar assembly 100 or the device receptacle 204 of the neck flange assembly 200. When the tracheal collar assembly 100 is secured around the neck of a patient, the device receptacle 104 and the associated monitoring device 340 will be positioned at or near a carotid artery of a patient. Similarly, when the neck flange assembly 200 is secured around the neck of a patient, the device receptacle 204 and the associated monitoring device 340 will be positioned at or near a carotid artery of a patient. The monitoring device 340 can communicate with the neck of the patient through the one or more apertures 130, 132, 134 through the bottom surface 128 of the device receptacle 104 of the collar body 102. Similarly, the monitoring device 340 can communicate with the neck of the patient through the one or more apertures 230, 232, 234 through the bottom surface 228 of the device receptacle 204 of the flange base 202. Thus, the monitoring device will be positioned near the carotid artery of the patient in order to measure the physiological parameters of the patient.

The monitoring device 340 can be detached or removed from either the device receptacle 104 of the collar body 102, the device receptacle 204 of the flange base 202, or the device receptacle of the flange on the tracheostomy tube body. Thus, the monitoring device can be removed so that the collar body 102, flange base 202, or flange on the tracheostomy tube body can be cleaned and disinfected.

The monitoring device 340 may contain one or more sensors. The one or more sensors may include a piezoelectric sensor, a photoplethysmographic sensor, an infrared sensor, an optoelectronic sensor, and a temperature sensor. The one or more sensors may be configured to measure temperature, heart rate, respiratory rate, blood pressure, and blood oxygen saturation. In one embodiment, the monitoring device 340 includes a piezoelectric sensor 342, a temperature sensor 344, and an infrared (IR) sensor 346. The piezoelectric sensor 342 is operable to detect heart rate, respiratory rate, and potentially blood pressure. The one or more piezoelectric sensor may be a single sensor or may be an array of multiple sensors. In some embodiments, physiological parameters may be calculated from one or more sensor measurements. In some examples, a sensor may be configured to determine when a ventilator is connected or disconnected. In another example, a sensor may be a pulse oximeter. In other examples, a sensor may be configured to detect a disconnection between the tracheostomy tube and the collar or neck flange. In one aspect, a controller 348 is operable to calculate physiological parameters based on one or more sensor measurements. In other aspects, an external processor is operable to calculate physiological parameters based on one or more sensor measurements.

The monitoring device may include a controller 348 operable to communicate the measurements from the one or more sensors to a secondary device. The controller 348 is in electrical communication with the one or more sensors. In some embodiments, the controller 348 may be a microcontroller. The controller 348 may contain a wireless transceiver operable to wirelessly communicate via radio frequency, (RF), Wi-Fi, or Bluetooth. In one embodiment, the controller 348 is a Bluetooth LE module with MCU. Thus, the sensors can detect physiological parameters of the patient and can connect via Bluetooth to a secondary device, such as a smartphone. The app receiving the information may record historic physiological measurements that can be remotely accessed by the user caregiver, clinicians, and healthcare provider to determine next treatment steps. Additionally, the app receiving the information may have an alarm to alert the user caregiver, clinicians, and healthcare provider of measurements outside of pre-set thresholds.

The monitoring device 340 may include a battery 352 to power the sensors. In one embodiment, the sensors are powered by a Lithium-Ion battery (i.e., a Li-ion battery). For example, the sensors may be powered by a Lithium Polymer battery (i.e., a LiPo battery). The monitoring device 340 may also include a charging module 354 operable to charge or recharge the battery 352. In one embodiment, the charging module 354 is a USB-C charging module. In other embodiments, the charging module can be a USB-A, USB-B, USB-B Mini, USB-B Micro, or 8-Pin Lightning charging module. Moreover, the monitoring device 340 may include voltage regulator 350. In one embodiment, the voltage regulator 350 may be configured to regulate voltage from 3.7 to 5V between the battery 352 and the controller 348.

### EXAMPLES

### Example 1

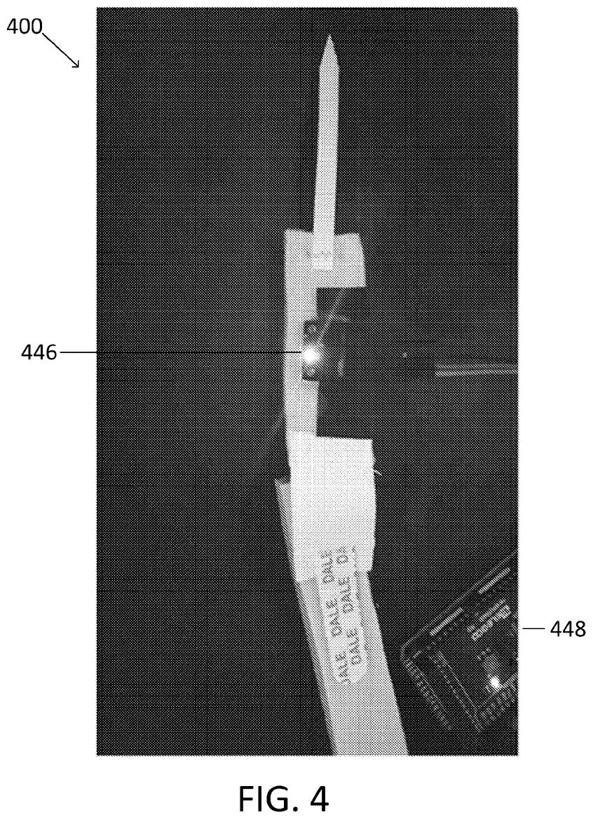

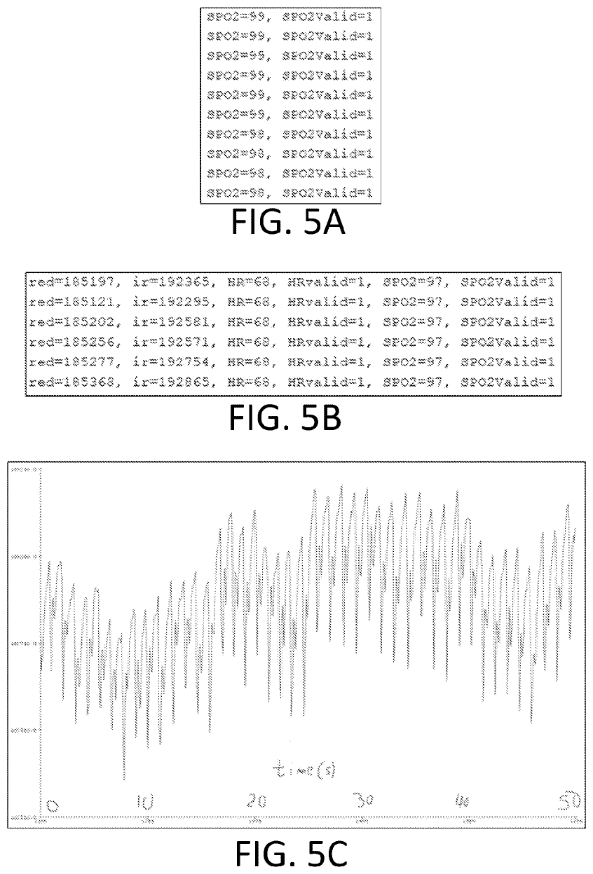

FIG. 4 is a photograph of a tracheal collar assembly 400 with an infrared (IR) sensor 446 connected to an Arduino microcontroller 448. FIG. 5A illustrates sample pulse oximetry data from the sensor and collar placed on skin. Sample data from the IR sensor on the collar, including heart rate and pulse oximetry, is shown in FIG. 5B. FIG. 5C shows waveform data from the sensor on the collar, showing heart rate with similar appearance to an electrocardiogram.

### EXEMPLARY EMBODIMENTS

The following is a list of non-limiting exemplary embodiments and may include combinations thereof.

Embodiment 1: A system for monitoring a patient with a tracheostomy tube, the system comprising: a collar comprising a device receptacle, wherein the collar is operable to stabilize the tracheostomy tube of the patient when the collar is secured around a neck of the patient; and a monitoring device operable to be inserted into the device receptacle of the collar, the monitoring device comprising a microcontroller and one or more sensors, the one or more sensors operable to monitor one or more physiological parameters of the patient when the monitoring device is inserted into the device receptacle of the collar.

Embodiment 2: The system of embodiment 1, wherein the collar is adjustable to secure around various neck sizes, and the device receptacle of the collar positions the one or more sensors near a carotid artery of the patient when the collar is secured around the neck of the patient.

Embodiment 3: The system of embodiment 1, wherein the one or more physiological parameters of the patient are at least one of temperature, heart rate, respiratory rate, blood pressure, and blood oxygen saturation.

Embodiment 4: The system of embodiment 1, wherein the one or more sensors is at least one of a piezoelectric sensor, an optoelectronic sensor, a photoplethysmographic sensor, an infrared sensor, a pulse oximeter, and a temperature sensor.

Embodiment 5: The system of embodiment 1, wherein the microcontroller is

operable to communicate the one or more physiological parameters to a secondary device.

Embodiment 6: The system of embodiment 5, wherein the microcontroller further comprises a wireless transceiver operable for wireless communication including at least one of radio frequency (RF), Wi-Fi, or Bluetooth.

Embodiment 7: The system of embodiment 1, wherein the monitoring device further comprises a rechargeable battery.

Embodiment 8: The system of embodiment 1, wherein the collar is made of an elastomer.

Embodiment 9: The system of embodiment 8, wherein the elastomer is silicone.

Embodiment 10: A system for monitoring a patient with a tracheostomy tube, the system comprising: a neck flange comprising a device receptacle and a void, wherein the neck flange is operable to contact the neck of a patient and to allow a proximal end of the tracheostomy tube of the patient to pass through the void in the neck flange; a tracheostomy collar operable to connect to each end of the neck flange and secure around the neck of the patient, wherein the tracheostomy collar maintains the position of the neck flange and the neck flange stabilizes the tracheostomy tube of the patient; and a monitoring device operable to be inserted into the device receptacle of the neck flange, the monitoring device comprising a microcontroller and one or more sensors, the one or more sensors operable to monitor one or more physiological parameters of the patient when the monitoring device is inserted into the device receptacle of the neck flange.

Embodiment 11: The system of embodiment 10, wherein the tracheostomy collar is adjustable to secure around various neck sizes, and the device receptacle of the neck flange positions the one or more sensors near a carotid artery of the patient when the tracheostomy collar is secured around the neck of the patient.

Embodiment 12: The system of embodiment 10, wherein the one or more physiological parameters of the patient are at least one of temperature, heart rate, respiratory rate, blood pressure, and blood oxygen saturation.

Embodiment 13: The system of embodiment 10, wherein the one or more sensors is at least one of a piezoelectric sensor, a photoplethysmographic sensor, an optoelectronic sensor, an infrared sensor, a pulse oximeter, and a temperature sensor.

Embodiment 14: The system of embodiment 10, wherein the microcontroller is operable to communicate the one or more physiological parameters to a secondary device.

Embodiment 15: The system of embodiment 14, wherein the microcontroller further comprises a wireless transceiver operable for wireless communication including at least one of radio frequency (RF), Wi-Fi, or Bluetooth.

Embodiment 16: The system of embodiment 10, wherein the monitoring device further comprises a rechargeable battery.

Embodiment 17: The system of embodiment 10, wherein the tracheostomy collar is made of an elastomer.

Embodiment 18: The system of embodiment 17, wherein the elastomer is silicone.

Embodiment 19: A tracheostomy tube comprising: a tube body; a flange extending radially from the tube body having a device receptacle, wherein the flange is operable to contact a neck of a patient; and a monitoring device operable to be inserted into the device receptacle, the monitoring device having a microcontroller and one or more sensors, wherein the one or more sensors are operable to monitor one or more physiological parameters of the patient.

Embodiment 20: The tracheostomy tube of embodiment 19, wherein the flange is adjustable to secure around various neck sizes and the sensors are positioned near a carotid artery of the patient when the flange is secured around the neck of the patient.

Embodiment 21: The tracheostomy tube of embodiment 19, wherein the one or more physiological parameters of the patient are at least one of temperature, heart rate, respiratory rate, blood pressure, and blood oxygen saturation.

Embodiment 22: The tracheostomy tube of embodiment 19, wherein the one or more sensors is at least one of a piezoelectric sensor, a photoplethysmographic sensor, an optoelectronic sensor, an infrared sensor, a pulse oximeter, and a temperature sensor.

Embodiment 23: The tracheostomy tube of embodiment 19, wherein the microcontroller is operable to communicate the one or more physiological parameters to a secondary device.

Embodiment 24: The tracheostomy tube of embodiment 23, wherein the microcontroller further comprises a wireless transceiver operable for wireless communication including at least one of radio frequency (RF), Wi-Fi, or Bluetooth.

Embodiment 25: The tracheostomy tube of embodiment 19, wherein the monitoring device further comprises a rechargeable battery.

Embodiment 26: A method of monitoring a patient with a tracheostomy tube, the method comprising: inserting a monitoring device into a device receptacle of a tracheostomy collar, the monitoring device comprising a microcontroller and one or more sensors; securing the tracheostomy collar around a neck of the patient to stabilize the tracheostomy tube of the patient; monitoring, via the one or more sensors, one or more physiological parameters of the patient; and communicating, via the microcontroller, the one or more physiological parameters to a secondary device.

Embodiment 27: The method of embodiment 26, wherein the one or more physiological parameters are respiratory rate, blood oxygen saturation, heart rate, blood pressure and temperature.

Embodiment 28: The method of embodiment 26, the method further comprising providing an alarm when one or more physiological parameters exceed a threshold.

Embodiment 29: The method of embodiment 26, the method further comprising analyzing historic data from the physiological parameters to determine further treatment options.

Embodiment 30: The method of embodiment 26, wherein the microcontroller communicates the one or more physiological parameters via a wireless transceiver operable for wireless communication including at least one of radio frequency (RF), Wi-Fi, or Bluetooth

Embodiment 31: The method of embodiment 26, wherein the one or more sensors is at least one of a piezoelectric sensor, an optoelectronic sensor, a photoplethysmographic sensor, an infrared sensor, a pulse oximeter, and a temperature sensor.

Embodiment 32: The method of embodiment 26, wherein the monitoring device comprises a rechargeable battery.

Embodiment 33: A system for monitoring a patient with a tracheostomy tube, the system comprising: a securement apparatus comprising a device receptacle, wherein the securement apparatus is operable to stabilize the tracheostomy tube of the patient when the securement apparatus is secured around a neck of the patient; and a monitoring device operable to be inserted into the device receptacle of the securement apparatus, the monitoring device comprising a microcontroller and one or more sensors, the one or more sensors operable to monitor one or more physiological parameters of the patient when the device is inserted into the device receptacle of the collar.

Embodiment 34: The system of embodiment 33, wherein the securement apparatus is a collar, the collar comprising: a first section having at least one aperture; a second section having a boss, wherein the device receptacle is located on the second section adjacent to the boss of the second section; and a third section having at least one aperture and at least one boss, wherein the at least one aperture of the first section is configured to removably couple to the boss of the third section and the at least one aperture of the third section is configured to removably couple to the boss of the second section.

Embodiment 35: The system of embodiment 33, wherein the securement apparatus is adjustable to secure around various neck sizes, and the device receptacle of the securement apparatus positions the one or more sensors near a carotid artery of the patient when the securement apparatus is secured around the neck of the patient.

Embodiment 36: The system of embodiment 33, wherein the securement apparatus is a neck flange, the neck flange comprising: two apertures on opposite sides of the device receptacle; and a void operable to allow a proximal end of the tracheostomy tube of the patient to pass through the void in the neck flange, wherein the two apertures are each operable to receive a collar configured to secure the neck flange around the neck of the patient.

Embodiment 37: The system of embodiment 33, wherein the securement apparatus is a flange extending radially from a tube body of the tracheostomy tube, wherein the flange is operable to contact a neck of a patient.

Embodiment 38: The system of embodiment 33, wherein the one or more physiological parameters of the patient are at least one of temperature, heart rate, respiratory rate, blood pressure, and blood oxygen saturation.

Embodiment 39: The system of embodiment 33, wherein the one or more sensors is at least one of a piezoelectric sensor, an optoelectronic sensor, a photoplethysmographic sensor, an infrared sensor, a pulse oximeter, and a temperature sensor.

Embodiment 40: The system of embodiment 33, wherein the microcontroller is operable to communicate the one or more physiological parameters to a secondary device.

Embodiment 41: The system of embodiment 33, wherein the microcontroller further comprises a wireless transceiver operable for wireless communication including at least one of radio frequency (RF), Wi-Fi, or Bluetooth.

Embodiment 42: The system of embodiment 33, wherein the monitoring device further comprises a rechargeable battery.

Embodiment 43: The system of embodiment 33, wherein the securement apparatus is made of an elastomer. Embodiment 44: The system of embodiment 33, wherein the elastomer is

silicone.

Embodiment 45: A tracheostomy tube comprising: a tube body; a flange extending radially from the tube body having a device receptacle, wherein the flange is operable to contact a neck of a patient; and a monitoring device operable to be inserted into the device receptacle, the monitoring device having a microcontroller and one or more sensors, wherein the one or more sensors are operable to monitor one or more physiological parameters of the patient.

What is claimed is:

1. A system for monitoring a patient with a tracheostomy tube, the system comprising:
a securement apparatus comprising a device receptacle, wherein the securement apparatus is operable to stabilize the tracheostomy tube of the patient when the securement apparatus is secured around a neck of the patient; and
a monitoring device operable to be inserted into the device receptacle of the securement apparatus, the monitoring device comprising a microcontroller and one or more sensors, the one or more sensors operable to monitor one or more physiological parameters of the patient when the monitoring device is inserted into the device receptacle of the securement apparatus.

2. The system of claim 1, wherein the securement apparatus is a collar, the collar comprising:
a first section having at least one aperture;
a second section having a boss, wherein the device receptacle is located on the second section adjacent to the boss of the second section; and
a third section having at least one aperture and at least one boss,
wherein the at least one aperture of the first section is configured to removably couple to the boss of the third section and the at least one aperture of the third section is configured to removably couple to the boss of the second section.

3. The system of claim 1, wherein the securement apparatus is adjustable to secure around various neck sizes, and the device receptacle of the securement apparatus positions the one or more sensors near a carotid artery of the patient when the securement apparatus is secured around the neck of the patient.

4. The system of claim 1, wherein the securement apparatus is a neck flange, the neck flange comprising:
two apertures on opposite sides of the device receptacle; and
a void operable to allow a proximal end of the tracheostomy tube of the patient to pass through the void in the neck flange,
wherein the two apertures are each operable to receive a collar configured to secure the neck flange around the neck of the patient.

5. The system of claim 1, wherein the securement apparatus is a flange extending radially from a tube body of the tracheostomy tube, wherein the flange is operable to contact the neck of a patient.

6. The system of claim 1, wherein the one or more physiological parameters of the patient are at least one of temperature, heart rate, respiratory rate, blood pressure, and blood oxygen saturation.

7. The system of claim 1, wherein the one or more sensors is at least one of a piezoelectric sensor, an optoelectronic sensor, a photoplethysmographic sensor, an infrared sensor, a pulse oximeter, and a temperature sensor.

8. The system of claim 1, wherein the microcontroller is operable to communicate the one or more physiological parameters to a secondary device.

9. The system of claim 5, wherein the microcontroller further comprises a wireless transceiver operable for wireless communication including at least one of radio frequency (RF), Wi-Fi, or Bluetooth.

10. The system of claim 1, wherein the monitoring device further comprises a rechargeable battery.

11. The system of claim 1, wherein the securement apparatus is made of an elastomer.

12. The system of claim 11, wherein the elastomer is silicone.

13. A method of monitoring a patient with a tracheostomy tube, the method comprising:
inserting a monitoring device into a device receptacle of a tracheostomy collar, the monitoring device comprising a microcontroller and one or more sensors;
securing the tracheostomy collar around a neck of the patient to stabilize the tracheostomy tube of the patient;
monitoring, via the one or more sensors, one or more physiological parameters of the patient; and
communicating, via the microcontroller, the one or more physiological parameters to a secondary device.

14. The method of claim 13, wherein the one or more physiological parameters are respiratory rate, blood oxygen saturation, heart rate, blood pressure and temperature.

15. The method of claim 13, the method further comprising providing an alarm when one or more physiological parameters exceed a threshold.

16. The method of claim 13, the method further comprising analyzing historic data from the one or more physiological parameters to determine further treatment options.

17. The method of claim 13, wherein the microcontroller communicates the one or more physiological parameters via a wireless transceiver operable for wireless communication including at least one of radio frequency (RF), Wi-Fi, or Bluetooth.

18. The method of claim 13, wherein the one or more sensors is at least one of a piezoelectric sensor, an optoelectronic sensor, a photoplethysmographic sensor, an infrared sensor, a pulse oximeter, and a temperature sensor.

19. The method of claim 13, wherein the monitoring device comprises a rechargeable battery.

20. A tracheostomy tube comprising:
a tube body;
a flange extending radially from the tube body having a device receptacle, wherein the flange is operable to contact a neck of a patient;
and a monitoring device operable to be inserted into the device receptacle, the monitoring device having a microcontroller and one or more sensors, wherein the one or more sensors are operable to monitor one or more physiological parameters of the patient.
